(19) United States
(12) Patent Application Publication
(10) Pub. No.: US 2009/0253943 A1
(43) Pub. Date: Oct. 8, 2009

(54) BIRCH BARK PELLETIZATION AND METHODS FOR OBTAINING NATURAL PRODUCTS FROM BIRCH BARK PELLETS

(75) Inventors: Christian Edwardson, Duluth, MN (US); Christopher Shallice, Tampa, FL (US)

Correspondence Address: Myriad PHARMACEUTICALS, Inc.;c/o CPA Global, P.O. Box 52050, Minneapolis, MN, 55402, US

(73) Assignee: Myriad Genetics, Incorporated, Salt Lake City, UT (US)

(21) Appl. No.: 12/395,139

(22) Filed: Feb. 27, 2009

Related U.S. Application Data
(63) Continuation of application No. PCT/US2007/018988, filed on Aug. 29, 2006.
(60) Provisional application No. 60/840,737, filed on Aug. 29, 2006.

(51) Int. Cl. C07C 35/22; B29C 47/00; B32B 21/02

(52) U.S. Cl. 568/817; 264/334; 428/402

(57) ABSTRACT

The present invention provides birch bark pellets that include outer birch bark. The present invention also provides methods of manufacturing birch bark pellets from outer birch bark. The present invention also provides methods for obtaining a natural product from birch bark pellets. The birch bark pellets are manufactured from outer birch bark that includes relatively low amounts of inner birch bark and wood. The birch bark pellets meet the requirement for extraction, to obtain e.g., betulin and/or lupeol from the pellets.

### CROSS-REFERENCE TO RELATED APPLICATIONS

This application is a continuation of PCT application No. PCT/US07/018,988, filed Aug. 29, 2007, which claims benefit of 60/840,737, filed Aug. 29, 2006 both of which are hereby incorporated by reference in their entirety.

### BACKGROUND OF THE INVENTION

Birch bark is a low-value waste product in the forest products industry today. Ekman, R., Holzforschung, (1983) 37, 205. Thus, vast quantities of birch bark and its chemical components are available.

Birch bark is a potential source of a variety of organic chemicals. Several triterpenoids have been identified in birch bark extracts. For example, lupeol, betulin, betulinic aldehyde, betulinic acid, methyl betulinate, lupenone, betulonic aldehyde, betulonic acid, β-amyrin, erythrodiol, oleanolic aldehyde, oleanolic acid, methyl linoleate, and acetyl oleanolic acid are all present in the outer bark of Betula verrucosa. Eckerman, C., (1985) Paperi ja Puu, No. 3, 100. In addition, several suberinic acids isolated from birch bark, as well as several triterpenoids, have been identified in the bark of Betula verrucosa. Ekman, R., Holzforschung, (1983) 37, 205.

The chemical constituents of birch bark are useful in pharmaceutical and industrial applications. For example, U.S. Pat. No. 5,750,578 discloses that betulin possesses antiviral properties and is useful to treat herpes virus. Betulin also possesses anti-feedant activity against boll weevils, and anti-inflammatory activity Miles, D. H., 1994, J. Agric. Food. Chem., 42, 1561-1562 and Recio, M., Planta Med., 1995, 61, 9-12. In addition, betulin showed cough suppressant and expectorant effects. Jinuhua, W., Zhongguo Yaoxue Zazhi, (1994), 29(5), 268-71. Betulin is also a useful starting material for preparing allobetulin and derivatives thereof, which possess useful pharmacological properties.

Betulin can be converted to betulinic acid, which is useful as a therapeutic agent. For example, Pisha, E. et al., (1995) J. M. Nature Medicine, 1, 1046-1051 discloses that betulinic acid has anti-tumor activity against human melanoma, e.g., MEL-1, MEL-2 and MEL-4. In addition, Fujioka, T. et al., J. Nat. Prod., (1994) 57, 243-247 discloses that betulinic acid has anti-HIV activity in H9 lymphocytic cells.

Current methods for isolating the chemical constituents of birch bark are deficient in several ways. For example, betulin has been extracted from the bark of white-barked birches in amounts up to 20%, based on the dry weight of the bark. Ekman, R., (1983) Holzforschung, 37, 205, Ohara, S., et al., (1986) Mokuza Gakkaishi, 32, 266. In addition, betulin has been isolated from outer birch bark waste of Betula verrucosa by liquid extraction employing boiling organic solvents and subsequent recrystallization. Eckerman, C., (1985) Paperi ja Puu, No. 3, 100. While current processes afford acceptable yields of betulin (e.g., 11-20%); these processes suffer from several major drawbacks. Specifically, the current methods employed to isolate betulin and other components in birch bark (e.g., lupeol and betulinic acid) are costly, inefficient, and unsafe.

Russian Patent Nos. RU2175326 (publication date 27 Oct. 2001) and RU2192879 (publication date 20 Nov. 2002), disclose methods of preparing betulin and derivatives thereof. The methods disclosed in Russian Patent No. RU2192879 include birch bark milling, separation of birch bark fibers, extraction of birch bark, separation of solution from extracted birch bark, and solvent removal from solution.

U.S. Patent Application Publication No. 2003/0153776 A1 (the '776 patent application), assigned to Boehringer Ingelheim Pharma, discloses a process for obtaining betulin from birch bark (see Abstract). The process includes extracting birch bark with a high-boiling, water-immiscible solvent, and extracting this extract with a dilute aqueous base (see claim 1). The methods in the '776 patent application are disclosed to provide betulin. Only 4 weight percent (wt. %) of betulin and no other triterpenoids (e.g., lupeol, betulinic acid, or a combination thereof) are obtained with the methods described therein. The use of charcoal is also required in the methods described therein. It is believed that the use of a decolorization agent such as activated charcoal decreases the overall yield of betulin. The methods described in the '776 patent application are not able to effectively remove triterpenoids such as betulin-3-caffeate, betulinic acid, lupeol, esters of fatty acids, fatty acids, polyphenols, and tannins, from the birch bark or birch bark extract. The process described in the '776 patent application is unsuitable for the industrial scale recovery of betulin, lupeol, and betulinic acid. The yields and purities of betulin disclosed in the '776 patent application are believed to be erroneous based on use of the processes described therein. Even if accurate, the yields and purities of betulin disclosed in the '776 patent application can be improved. The processes described therein also cannot be practiced on larger industrial scales (e.g., 700 kilograms).

U.S. Pat. Nos. 6,232,481, 6,271,405, 6,867,314, and 6,407,270 describe methods for manufacturing betulinic acid from betulin. U.S. Pat. Nos. 6,392,070, 6,634,575, and 6,815,553 describe birch bark processing and the isolation of natural products (e.g., betulin) from birch bark. U.S. Published Patent Application No. 2004/0009242 describes processes for extracting compounds (e.g., betulin) from plants (e.g., birch). U.S. Pat. No. 6,768,016 describes the isolation of natural products (e.g., betulin) from birch bark.

A need therefore exists for safer, more cost-effective and/or more efficient methods to obtain commercial quantities (e.g., tons) of betulin, as well as commercial quantities (e.g., kilograms) of lupeol and betulinic acid from birch bark.

### SUMMARY OF INVENTION

The present invention provides birch bark pellets that include at least about 70 wt. % outer birch bark. The present invention also provides methods of manufacturing birch bark pellets from outer birch bark. The present invention also provides methods for obtaining a natural product from birch bark pellets. The birch bark pellets are manufactured from outer birch bark that includes relatively low amounts of inner birch bark and wood. The birch bark pellets meet the requirement for extraction to obtain, for example, betulin and/or lupeol from the pellets. Compared to outer birch bark or to birch bark pellets formed from both inner and outer birch bark, use of the birch bark pellets on an industrial scale (e.g., 700 kilogram (kg)) reduces the shipping and processing costs, and/or increases the yield of natural product obtained. The process of obtaining the natural products can be performed in a continuous belt extractor at a temperature of about 95° C. (Centigrade). Utilizing outer birch bark in the form of pellets, and utilizing toluene as the solvent for the extraction, the yield was increased from about 60 wt. % to about 85 wt. %. Additionally, the amount of solvent employed was reduced from about 7 kg/kg of outer birch bark to about 5 kg/kg of outer birch bark.

The present invention also provides reformulated birch bark pellets that include birch bark and additional ingredients. The present invention also provides methods of manufacturing reformulated birch bark pellets from birch bark pellets. The present invention also provides methods for obtaining a natural product from reformulated birch bark pellets. The reformulated birch bark pellets are manufactured from outer birch bark that includes relatively low amounts of inner birch bark and wood. The reformulated birch bark pellets meet the requirement for extraction to obtain, for example, betulin and/or lupeol from the pellets.

The present invention provides a method of manufacturing birch bark pellets, the method including: (a) debarking a birch tree to provide birch bark; (b) separating the inner birch bark from the outer birch bark; (c) reducing the size of the outer birch bark; (d) conditioning the outer birch bark; and (e) extruding the outer birch bark, effective to provide birch bark pellets.

In one embodiment, the birch bark pellets comprise less than about 12 wt. % inner birch bark. In another embodiment, the birch bark pellets comprise at least about 70 wt. % outer birch bark. In yet another embodiment, the birch bark pellets comprise less than about 10 wt. % wood.

In one embodiment, the birch bark pellets comprise greater than about 5 wt. % free (extractable) betulin. In another embodiment, the birch bark pellets have a density of at least 0.4 grams/centimeter³ (gm/cm³). In yet another embodiment, the birch bark pellets comprise less than about 15 wt. % water.

In one embodiment, the birch bark pellets comprise less than about 5 wt. % water. In another embodiment, the birch bark pellets have a diameter of about ⅛ inch to about ¾ inch. In yet another embodiment, the birch bark pellets have a bulk density of from about 280 to about 420 grams/liter (gm/L).

In one embodiment, the debarking in step (a) is carried out employing a ring debarker, a drum debarker, or a Rosserhead debarker.

In one embodiment, the method includes, after the debarking in step (a), separating the birch bark from debris present in the birch bark. In another embodiment, the method includes, after the debarking in step (a), separating the birch bark from debris present in the birch bark employing a screen having openings of at least about ⅝ inch. In yet another embodiment, the method includes, after the debarking in step (a), separating the birch bark from debris present in the birch bark employing a screen having openings of about 1 inch or less.

In one embodiment, the method includes, after the debarking in step (a), separating the birch bark from debris present in the birch bark, wherein the debris includes at least one of rocks, metal and wood. In another embodiment, the method includes, after the debarking in step (a), separating the birch bark from debris present in the birch bark, wherein the debris includes wood having a diameter of ⅝ inch or smaller. In yet another embodiment, the method includes, after the debarking the birch tree to provide birch bark in step (a), reducing the size of the birch bark.

In one embodiment, the method includes, after the debarking the birch tree to provide birch bark in step (a), reducing the size of the birch bark, wherein the reducing the size of the birch bark in step (c) is carried out employing a hammermill or any other suitable method of mechanical reduction. In another embodiment, the method includes before separating the inner birch bark from the outer birch bark in step (b), aspirating the birch bark, effective to remove wood present in the birch bark. In yet another embodiment, the separating the inner birch bark from the outer birch bark in step (b) includes subjecting the birch bark to fragmentation to provide a combination of outer birch bark and inner birch bark and separating the outer birch bark from the inner birch bark.

In one embodiment, the separating the inner birch bark from the outer birch bark in step (b) includes subjecting the birch bark to fragmentation to provide a combination of outer birch bark and inner birch bark and separating the outer birch bark from the inner birch bark by passing the inner birch bark through a mesh effective to separate the outer birch bark from the inner birch bark. In another embodiment, the separating the inner birch bark from the outer birch bark in step (b) includes subjecting the birch bark to fragmentation with a chipper or a shredder to provide a combination of outer birch bark and inner birch bark and separating the outer birch bark from the inner birch bark.

In one embodiment, the separating the inner birch bark from the outer birch bark in step (b) further includes separating wood from the outer birch bark. In another embodiment, the separating the inner birch bark from the outer birch bark in step (b) is carried out employing a straight line screener, a deck screen, a rotary screen, a vibratory screen, a shaker screen, or a trommel screen. In another embodiment, the reducing the size of the outer birch bark in step (c) is carried out employing a rotary cutter, a knife cutter, a hammermill, a granulator, or any other suitable method of mechanical reduction.

In one embodiment, the conditioning the outer birch bark in step (d) includes contacting the outer birch bark with at least one of steam and water. In another embodiment, the extruding the outer birch bark in step (e) is carried out employing a die having an orifice with a diameter of about ⅛ inch to about ¾ inch. In yet another embodiment, the extruding the outer birch bark in step (e) is carried out employing a die having an average temperature of about 100° F. (Fahrenheit) to about 250° F.

In one embodiment, the method further includes, after the extruding in step (e), cooling the birch bark pellets. In another embodiment, the method further includes, after the extruding in step (e), cooling the birch bark pellets to about 125° F. to about 130° F. In yet another embodiment, the method includes, after the extruding in step (e), packaging the birch bark pellets.

In one embodiment, at least about 15 kg of the birch bark pellets are obtained, per about 100 kg of birch bark employed.

The present invention also provides a birch bark pellet including at least about 70 wt. % outer birch bark. In one embodiment, the birch bark pellet includes less than about 12 wt. % inner birch bark. In another embodiment, the birch bark pellet includes less than about 10 wt. % wood. In yet another embodiment, the birch bark pellet includes up to about 20 wt. % free (extractable) betulin.

In one embodiment, the birch bark pellet includes greater than about 5 wt. % free (extractable) betulin. In another embodiment, the birch bark pellets have a density of at least 0.4 gm/cm³. In yet another embodiment, the birch bark pellet includes less than about 15 wt. % water. In one embodiment, the birch bark pellet includes less than about 5 wt. % water.

In one embodiment, the birch bark pellet has a diameter of about ⅛ inch to about ¾ inch.

The present invention also provides a method of manufacturing reformulated birch bark pellets, the method including: (a) grinding the birch bark pellets; (b) blending the ground birch bark pellets with an inert fibrous material, a mineral composition, an alkaline composition, and an optional solvent to afford a blended birch bark composition; and (c) extruding the blended birch bark composition to provide reformulated birch bark pellets.

In one embodiment, the inert fibrous material includes de-oiled fibrous plant material. In another embodiment, the de-oiled fibrous plant material is cellulose, lignin, wood, cotton, hemp jute, flax, ramie, sisal, spent sunflower meal, rice hulls, oat hulls, or a combination thereof. In yet another embodiment, the de-oiled fibrous plant material is spent sunflower meal.

In one embodiment, the mineral composition includes a metal carbonate. In another embodiment, the metal carbonate is lime.

In one embodiment, the alkaline composition includes an alkali metal hydroxide, an alkaline earth metal hydroxide, or a combination thereof. In another embodiment, the alkali metal hydroxide is sodium hydroxide (NaOH).

In one embodiment, the optional solvent includes an aqueous solvent, an organic solvent, or a combination thereof. In another embodiment, the aqueous solvent is water. In yet another embodiment, there is no solvent.

In one embodiment, the reformulated birch bark pellets comprise less than about 6 wt. % inner birch bark. In another embodiment, the reformulated birch bark pellets comprise at least about 30 wt. % outer birch bark. In yet another embodiment, the reformulated birch bark pellets comprise less than about 5 wt. % wood.

In one embodiment, the reformulated birch bark pellets comprise greater than about 2 wt. % free (extractable) betulin. In another embodiment, the reformulated birch bark pellets have a density of at least 0.4 gm/cm³. In yet another embodiment, the reformulated birch bark pellets have a diameter of about ⅛ inch to about ¾ inch.

The present invention also provides a reformulated birch bark pellet including ground birch bark with an inert fibrous material, a mineral composition, an alkaline composition, and an optional solvent.

In one embodiment, the inert fibrous material includes de-oiled fibrous plant material. In another embodiment, the de-oiled fibrous plant material is cellulose, lignin, wood, cotton, hemp jute, flax, ramie, sisal, spent sunflower meal, rice hulls, oat hulls, or a combination thereof. In yet another embodiment, the de-oiled fibrous plant material is spent sunflower meal.

In one embodiment, the mineral composition includes a metal carbonate. In another embodiment, the metal carbonate is lime.

In one embodiment, the alkaline composition includes an alkali metal hydroxide, an alkaline earth metal hydroxide, or a combination thereof. In another embodiment, the alkali metal hydroxide is sodium hydroxide (NaOH).

In one embodiment, the optional solvent includes an aqueous solvent, an organic solvent, or a combination thereof. In another embodiment, the aqueous solvent is water. In yet another embodiment, there is no solvent.

In one embodiment, the reformulated birch bark pellet includes at least about 30 wt. % outer birch bark. In another embodiment, the reformulated birch bark pellet includes less than about 6 wt. % inner birch bark. In yet another embodiment, the reformulated birch bark pellet includes less than about 5 wt. % wood.

In one embodiment, the reformulated birch bark pellet includes up to about 9 wt. % free (extractable) betulin. In another embodiment, the reformulated birch bark pellet includes greater than about 2 wt. % free (extractable) betulin.

In one embodiment, the reformulated birch bark pellet has a density of at least 0.4 gm/cm³. In another embodiment, the reformulated birch bark pellet has a diameter of about ⅛ inch to about ¾ inch.

The present invention also provides a method of manufacturing reformulated birch bark pellets, the method including: (a) grinding the birch bark pellets, an inert fibrous material, a mineral composition, an alkaline composition, and an optional solvent to afford a birch bark composition; and (b) extruding the birch bark composition to provide reformulated birch bark pellets.

The present invention also provides a method for obtaining a natural product from birch bark pellets, the method including: (a) contacting birch bark pellets with an organic solvent to provide a birch bark extract; (b) contacting the birch bark extract with an aqueous base to provide an organic phase and an aqueous phase; (c) separating the organic phase from the aqueous phase; and (d) separating solids from the organic phase to provide the natural product.

In one embodiment, at least about 700 kg of birch bark pellets are employed. In another embodiment, the natural product includes betulin, lupeol, or a combination thereof. In yet another embodiment, at least about 1675 kg of betulin is obtained, per about 18850 kg of birch bark pellets employed.

In one embodiment, the organic solvent includes toluene. In another embodiment, the organic solvent includes 10 kg of toluene per about 1 kg of birch bark pellets, or less.

In one embodiment, the contacting of the birch bark pellets with the organic solvent is carried out for about 3 to about 5 hours. In another embodiment, the contacting of the birch bark pellets with the organic solvent is carried out under an inert atmosphere. In yet another embodiment, the contacting of the birch bark pellets and the organic solvent is carried out under nitrogen.

In one embodiment, the contacting of the birch bark pellets with the organic solvent is carried out at a temperature of about 65° C. to about 105° C. In another embodiment, the contacting of the birch bark pellets with the organic solvent is carried out while agitating the birch bark pellets and the organic solvent.

In one embodiment, the method further includes, after the contacting of the birch bark pellets with the organic solvent in step (a), removing at least a portion of the organic solvent.

In one embodiment, the aqueous base includes an alkaline metal or an alkaline earth metal. In another embodiment, the aqueous base includes sodium hydroxide (NaOH), potassium hydroxide (KOH), or a combination thereof. In yet another embodiment, the aqueous base includes 7.5% aqueous sodium hydroxide (NaOH).

In one embodiment, in step (b), the contacting the birch bark extract with the aqueous base is carried out at a temperature and for a period of time effective to hydrolyze natural esters of the natural product.

In one embodiment, the method further includes, after the contacting the birch bark extract with the aqueous base in step (b), separating the birch bark extract and the aqueous base. In another embodiment, the method further includes, after the contacting the birch bark extract with the aqueous base in step (b), separating the birch bark extract and the aqueous base and cooling the birch bark extract. In yet another embodiment, the method further includes, after the contacting the birch bark extract with the aqueous base in step (b), separating the birch bark extract and the aqueous base and cooling the birch bark extract to below about 80° C.

In one embodiment, the method further includes, after the contacting the birch bark extract with the aqueous base in step (b), separating the birch bark extract and the aqueous base, and crystallizing betulin from the extract. In another embodiment, the method further includes, after the contacting the birch bark extract with the aqueous base in step (b), separating the birch bark extract and the aqueous base, crystallizing betulin from the extract, and washing the betulin with an organic solvent. In yet another embodiment, the method further includes, after the contacting the birch bark extract with the aqueous base in step (b), separating the birch bark extract and the aqueous base, crystallizing betulin from the extract, and washing the betulin with toluene having a temperature of at least about 65° C. to about 85° C.

In one embodiment, any of the birch bark pellets described above are employed. In another embodiment, any of the outer birch bark pellets described are employed.

In one embodiment, the method further includes, after the separating solids from the organic phase in step (d), condensing the mother liquor (filtrate) to provide a second natural product. In another embodiment, the second natural product is 3-acetylbetulin, 28-acetylbetulin, betulone, betulinic aldehyde, 3-acetyl betulinic aldehyde, betulinic acid, 3-acetyl betulinic acid, betulone, betulinic acid, 3-acetylupeol, lupine, allobetulin, 3-acetylallobetulin, allobetulone, betulin 3-caffeate, platanic acid, messagenic acid, allobetulin lactone, allobetulone lactone, 2,3-diketoallobetulin lactone, (7S,9R)-7-[(2S,4S,5R,6S)-4-amino-5-hydroxy-6-methyl-oxan-2-yl]oxy-6,9,11-trihydroxy-9-(2-hydroxyacetyl)-4-methoxy-8,10-dihydro-7H-tetracene-5,12-dione, dihydrobetulin, dihydrobetulone, dihydrobetulinic aldehyde, dihydrobetulonic aldehyde, dihydrobetulonic acid, dihydrobetulinic acid, dihydrolupeol, dihydrolupeone, diacetylbetulin, lupeol, or 3-acetyldihydrobetulin.

In one embodiment, the method further includes, after the contacting the birch bark extract with the aqueous base in step (b), separating the birch bark extract and the aqueous base, washing the birch bark extract at least one or more times with water, decanting the birch bark extract to remove water, reducing the organic solvent from the birch bark extract by evaporation, cooling the birch bark extract, crystallizing betulin from the birch bark extract, and washing the betulin with toluene.

In one embodiment, the contacting the birch bark extract with the aqueous base in step (b) uses a solvent:caustic ratio of about 5:1. In another embodiment, the washing the birch bark extract at least one or more times with water uses a solvent:water ratio of about 4:1. In yet another embodiment, the residual water content of the birch bark extract after decanting is less than about 1 wt. %. In one embodiment, the cooling of the birch bark extract is performed in a continuous cooler at about 10° C.

The present invention also provides a method for obtaining a natural product from reformulated birch bark pellets, the method including: (a) contacting reformulated birch bark pellets with an organic solvent to provide a birch bark extract, wherein the birch bark pellets comprise ground birch bark with an inert fibrous material, a mineral composition, an alkaline composition, and an optional solvent; (b) contacting the birch bark extract with an aqueous base to provide an organic phase and an aqueous phase; (c) separating the organic phase from the aqueous phase; and (d) separating solids from the organic phase to provide the natural product.

In one embodiment, the natural product includes betulin, lupeol, or a combination thereof. In another embodiment, the method further includes, after the contacting the birch bark extract with the aqueous base in step (b), separating the birch bark extract and the aqueous base, washing the birch bark extract at least one or more times with water, decanting the birch bark extract to remove water, reducing the organic solvent from the birch bark extract by evaporation, cooling the birch bark extract, crystallizing betulin from the birch bark extract, and washing the betulin with toluene. In yet another embodiment, the contacting the birch bark extract with the aqueous base in step (b) uses a solvent:caustic ratio of about 5:1.

In one embodiment, the washing the birch bark extract at least one or more times with water uses a solvent:water ratio of about 4:1. In another embodiment, the residual water content of the organic phase after decanting is less than about 1 wt. %. In yet another embodiment, the cooling of the organic phase is performed in a continuous cooler at about 10° C.

### BRIEF DESCRIPTION OF THE DRAWINGS

Embodiments of the invention may be best understood by referring to the following description and accompanying drawings, which illustrate such embodiments. In the drawings:

FIG. 1 illustrates a block flow diagram depicting the production of birch bark pellets from birch trees.

FIG. 2 illustrates a block flow diagram depicting the production of birch bark pellets from birch trees.

FIG. 3 illustrates a block flow diagram depicting the reformulation of birch bark pellets.

FIG. 4 illustrates a block flow diagram depicting the isolation of betulin from birch bark pellets, and subsequent production of betulinic acid from betulin.

FIG. 5 illustrates a block flow diagram depicting the isolation of betulin from reformulated birch bark pellets.

FIG. 6 illustrates a block flow diagram depicting the isolation of betulin from birch bark pellets, and subsequent production of betulinic acid from betulin.

### DEFINITIONS

As used herein, certain terms have the following meanings. All other terms and phrases used in this specification have their ordinary meanings as one of skill would understand. Such ordinary meanings may be obtained by reference to technical dictionaries, such as Hawley's Condensed Chemical Dictionary 11^{th} Edition, by Sax and Lewis, Van Nostrand Reinhold, New York, N.Y., 1987, and The Merck Index, 11^{th} Edition, Merck & Co., Rahway N.J. 1989.

As used herein, the term “and/or” means any one of the items, any combination of the items or all of the items with which this term is associated.

As used herein, the singular forms “a,” “an,” and “the” include plural reference unless the context clearly dictates otherwise. Thus, for example, a reference to “a formulation” includes a plurality of such formulations, so that a formulation of compound X includes formulations of compound X.

As used herein, the term “about” means a variation of 10 percent of the value specified, for example, about 50 percent carries a variation from 45 to 55 percent. For integer ranges, the term “about” can include one or two integers greater than and less than a recited integer.

As used herein, the term “acidifying” refers to the process of lowering the pH to below 7.0. For example, in one specific embodiment, the acidifying includes lowering the pH to below about 6.0. In another specific embodiment, the acidifying includes lowering the pH to below about 5.0. In yet another specific embodiment, the acidifying includes lowering the pH to below about 4.0.

As used herein, the term “agitating” refers to the process of putting a mixture into motion with a turbulent force. Suitable methods of agitating include, e.g., stirring, mixing, and shaking.

As used herein, the term “alkaline composition” refers to a composition with a pH greater than 7.0.

As used herein, the term “alkaline metal” refers to metals of Group IA of the Periodic Table of Elements, e.g., sodium (Na) and potassium (K).

As used herein, the term “alkaline earth metal” refers to metals of Group IIA of the Periodic Table of Elements, e.g., magnesium (Mg) and calcium (Ca).

As used herein, the term “aqueous base” refers to a solution of water, and a substance that produces OH⁻ ions in the aqueous solution. Specifically, the aqueous base can include water and at least one of a lithium ion (Li⁺), a sodium ion (Na⁺), a potassium ion (K⁺), a calcium ion (Ca²⁺), and a barium ion (Ba⁺). More specifically, the aqueous base can include water and at least one of sodium hydroxide (NaOH) and potassium hydroxide (KOH).

As used herein, the term “aromatic hydrocarbon” refers to a compound having at least one phenyl or naphthyl ring, wherein the compound contains carbon and hydrogen atoms. The aromatic hydrocarbon can optionally be substituted, e.g., with one or more groups selected from the group of alkyl (e.g., methyl), hydroxyl, halo, alkoxy, cyano, carboxyl, sulfonyl, and amino. Suitable specific aromatic hydrocarbons include, e.g., xylenes, o-xylene, m-xylene, p-xylene, toluene, benzene, and combinations thereof.

As used herein, the term “blending” refers to the mixing of two or more materials.

As used herein, the term “birch” refers to any of the several deciduous trees of the genus Betula. The birches comprise the family Betulaceae in the order Fagales. Birch trees include, for example, white birch, B. alba; sweet, black, or cherry birch, B. lenta; monarch birch, B. maximowicziana; dwarf or arctic birch, B. nana; Japanese white birch, B. platyphyla japonica; smooth-bark birch, B. pubescens; yellow birch, B. alleghaniensis; paper, white or canoe birch, B. papyrifera; grey birch, B. populifolia; river birch, B. nigra; and the European birches, B. pubescens; B. alba and B. pendula. Specifically, birch can be B. alba, B. lenta, B. maximowicziana, B. nana, B. platyphylajaponica, B. pubescens, B. alleghaniensis, B. papyrifera, B. populifolia, B. nigra, B. pubescens, B. alba, or B. pendula. A specific birch useful in the methods of the present invention may be B. papyrifera.

As used herein, the term “birch bark pellet” refers to a pellet that includes birch bark. It is appreciated that those of skill in the art understand that a birch bark pellet is typically characterized by those starting materials or intermediate components that are useful in making the birch bark pellets. While these materials may undergo a substantial conversion during the manufacturing of the birch bark pellet, reference to birch bark pellet as including these materials or various other components is acceptable and appropriate to those of skill in the art. For example, the birch bark and the other components during the conditioning and/or extruding steps, can undergo a chemical and/or physical conversion, such that they may no longer expressly and literally meet the criteria to be classified as birch bark and the various other components, respectively. Reference to the birch bark pellet as including birch bark and various other components is, however, acceptable and appropriate to those of skill in the art. As such, as used herein, “birch bark pellet” includes birch bark and various other components (e.g., an inert fibrous material, a mineral composition, an alkaline composition, and an optional solvent).

As used herein, the term “chipper” refers to a machine that reduces organic matter to smaller size pieces. For example, a chipper reduces whole tree trunks to sawdust.

As used herein, the terms “comprising,” “including,” “containing,” “characterized by,” and grammatical equivalents thereof are inclusive or open-ended terms that do not exclude additional, un-recited elements or method steps. “Comprising” is to be interpreted as including the more restrictive terms “consisting of” and “consisting essentially of”

As used herein, the term “concentrating” or “condensing” refers to the process whereby the volume of a substance is reduced, by the removal of liquid.

As used herein, the term “conditioning” refers to the process whereby the birch bark is subjected to treatment with water, heat, steam, or blended with other ingredients.

As used herein, the term “cord” refers to a unit of amount of wood cut for burning. A cord is typically about 128 cubic feet. Alternatively, the term “cord” may also refer to a string or a rope.

As used herein, the term “consisting of” and grammatical equivalents thereof exclude any element, step, or ingredient not specified in the claim.

As used herein, the term “consisting essentially of” and grammatical equivalents thereof limit the scope of a claim to the specified materials or steps and those that do not materially affect the basic and novel characteristic or characteristics of the claimed invention.

As used herein, the term “cooling” refers to the process whereby the temperature of an object is decreased.

As used herein, the term “debarking” refers to the process whereby the bark is removed from the tree.

As used herein, the term “deck screen” refers to a screen with one or more flat, usually rectangular, decks that hold a mesh of various materials and sizes such that the larger hole size is above a smaller hole size. A mixture of multiple size particles entering at the top can, by some form of agitation and the force of gravity, be separated into two or more parts with differing particle sizes.

As used herein, the term “decanting” refers to the process of pouring off a liquid without disturbing the sediment or the process of pouring off a liquid with a minimal disturbance of the sediment.

As used herein, the term “de-oiled fibrous plant material” refers to fibrous plant material that has had the natural plant oil removed.

As used herein, the term “distill” or “distillation” refers to the process of extracting the volatile components of a mixture by the condensation and collection of the vapors that are produced as the mixture is heated. The process includes the evaporation and subsequent collection of a liquid by condensation.

As used herein, the term “drying” refers to the removal of water and/or solvent, such that the water and/or solvent content is below about 5 wt. %, below about 2 wt. % or below about 1 wt. %.

As used herein, the term “drum debarker” refers to a machine that removes the bark from logs.

As used herein, the term “extruding” refers to the process whereby a material is forced through a die or opening to form a shape. In some cases, the material entering the extruder is less dense than the material exiting the extruder.

As used herein, the term “fragmentation” refers to chopping, crunching, crushing, gnashing, or pounding. Such fragmentation of birch bark will effectively provide inner birch bark, which consists of a small percentage of chunks and a large percentage of fines (i.e., material that can pass through a 20 and/or 60 mesh screen), and outer birch bark (e.g., in the form of shreds), which can be physically separated. The fragmentation can conveniently be carried out by feeding birch bark into a machine with knives on a rotating disk (e.g., a chipper, a shredder, or a hammermill).

As used herein, the term “filtering” refers to the process of removing solids from a mixture by passing the liquid through a filter, thereby suspending the solids on the filter.

As used herein, the term “hammermill” refers to a machine that includes a steel drum containing a vertical or horizontal cross-shaped rotor on which pivoting hammers are mounted. The hammers may be fixed or are free to swing on the ends of the cross. The rotor is spun at a high speed inside the drum while material is fed into a feed hopper. The bark is shredded by the impact of the hammers on the ends of the rotating cross against a screen (i.e., a metal plate with holes) through which the material exits the drum.

As used herein, the term “hydrolyze” or “hydrolysis” refers to the process of converting a carboxylic ester to the corresponding carboxylic acid, with the addition of water. The reaction (i.e., hydrolysis) can conveniently be carried out employing any suitable reagent(s) and reaction condition(s). For example, the reaction can be carried out in a suitable solvent and at a suitable temperature and pressure, under basic conditions, neutral conditions, or acidic conditions. Suitable reagents and reaction conditions are disclosed, e.g., in Advanced Organic Chemistry, Part B: Reactions and Synthesis, Carey and Sundberg, Plenum Press; Comprehensive Organic Transformations, Larock, Wiley & Sons; and Advanced Organic Chemistry, Reactions Mechanisms, and Structure, March, McGraw Hill.

For example, the hydrolysis can be carried out at a pH of greater than about 7.0 (e.g., a pH of about 7-8, 8-9, 9-10, 10-11, or 11-12). When the hydrolysis is carried out at a pH of greater than 7.0, one or more suitable bases will typically be employed. Suitable bases include metal hydroxides and metal alkoxides. Suitable metal hydroxides include sodium hydroxide, potassium hydroxide, lithium hydroxide, calcium hydroxide and barium hydroxide. Suitable metal alkoxides include lithium methoxide, lithium ethoxide, lithium isopropoxide, lithium tert-butoxide, sodium methoxide, sodium ethoxide, sodium isopropoxide, sodium tert-butoxide, potassium methoxide, potassium ethoxide, potassium isopropoxide, potassium tert-butoxide, magnesium methoxide, magnesium ethoxide, barium methoxide, barium ethoxide, calcium methoxide and calcium ethoxide. A specific base suitable for the processes of the present invention (i.e., hydrolysis of birch bark) is sodium hydroxide.

Alternatively, the hydrolysis can be carried out at a pH of less than about 7.0 (e.g., a pH of about 1-2, 2-3, 3-4, 4-5, 5-6, or 6-7). When the hydrolysis is carried out at a pH of less than about 7.0, one or more suitable acids will typically be employed. Suitable acids include, for example, hydrochloric acid, phosphoric acid, formic acid, hydrobromic acid, sulfuric acid, nitric acid, acetic acid, and combinations thereof. A specific acid suitable for the processes of the present invention (i.e., hydrolysis of birch bark) is sulfuric acid.

Alternatively, the hydrolysis can be carried out at a pH of about 6.5 to about 7.5 (i.e., neutral conditions). When the hydrolysis is carried out at about 6.5 to about 7.5 (i.e., neutral conditions), water at standard pressure or at an elevated pressure can be employed.

As used herein, the term “inert atmosphere” refers to an atmosphere of nitrogen gas and the noble gases (i.e., the elements in Group 18 (Group 0 IUPAC style) of the periodic table). Particularly suitable noble gases include helium and argon.

As used herein, the term “lime” refers to crushed limestone, which is a rock of marine and fresh-water sediments and is composed primarily of calcite (CaCO₂).

As used herein, the term “mesh” refers to the number of opening per inch of a screen and is used to determine the size of particles that can pass through the screen or be retained on the screen.

As used herein, the term “metal carbonate” refers to a metal salt of a carbonic acid. Suitable metal carbonates include, for example, calcium carbonate, magnesium carbonate, and the like.

As used herein, the term “metal hydride” refers to a binary compound of hydrogen and a metal. Suitable metal hydrides include e.g., lithium hydride (LiH), sodium hydride (NaH), potassium hydride (KH), calcium hydride (CaH₂), and lithium aluminum hydride (LiAlH₄).

As used herein, the term “mineral composition” refers to any naturally occurring inorganic material that has a definite chemical composition and characteristic physical properties.

As used herein, the term “organic solvent” refers to solvents that contain carbon.

As used herein, the term “pellet” refers to a symmetrical or asymmetrical compressed form of matter. For example, a pellet may be a sphere, a cylinder, or any other shaped object of compressed material. A pellet may be, for example, a bale, a puck, and the like.

As used herein, the term “pelletization” refers to the process of forming bark pellets (e.g., outer birch bark pellets).

As used herein, the term “natural ester” refers to an organic compound (e.g., triterpenes or triterpenoids) having at least one carboxylic ester group.

As used herein, the term “neutralizing” refers to the process of changing or bringing the pH to about 7±1. As such, the neutralizing can include bringing the pH to about 6 to about 8. In a specific embodiment, the neutralizing can include bringing the pH to about 6.5 to about 7.5 (i.e., to a pH of about 7±0.5). In yet another specific embodiment, the neutralizing can include bringing the pH to about 6.75 to about 7.25 (i.e., to a pH of about 7±0.25).

As used herein, the term “non-polar organic solvent” refers to an organic solvent having no measurable dipole. Specifically, it refers to an organic solvent having a dielectric constant of less than about 15, less than about 10, or between about 6 and about 10.

As used herein, the term “packaging” refers to the process whereby the birch bark pellets are loaded into containers.

As used herein, the term “precipitating” refers to the process of causing a solid substance (e.g., crystals) to be separated from a solution. The precipitating can include, e.g., crystallizing.

As used herein, the terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “polar organic solvent” refers to an organic solvent having a measurable dipole. Specifically, it refers to an organic solvent having a dielectric constant of at least about 15, at least about 20 or between about 20 and about 30.

As used herein, the term “purifying” refers to the process of ridding a solid substrate (e.g., crystals) of impurities. Suitable methods of purifying include, e.g., washing, recrystallizing, and drying.

As used herein, the term “reformulated birch bark pellet” refers to a birch bark pellet that has been ground up, blended with additional ingredients, and extruded into new pellets. For example, a reformulated birch bark pellet may include birch bark and various other components (e.g., an inert fibrous material, a mineral composition, an alkaline composition, and a solvent). It is appreciated that those of skill in the art understand that a reformulated birch bark pellet is typically characterized by those starting materials or intermediate components that are useful in making the reformulated birch bark pellets. While these materials may undergo a substantial conversion during the manufacturing of the reformulated birch bark pellets, reference to reformulated birch bark pellet as including these materials or various other components is acceptable and appropriate to those of skill in the art. For example, the birch bark and the other components (e.g., an inert fibrous material, a mineral composition, an alkaline composition, and an optional solvent), during the conditioning and/or extruding steps, can undergo a chemical and/or physical conversion, such that they may no longer expressly and literally meet the criteria to be classified as birch bark and the various other components (e.g., an inert fibrous material, a mineral composition, an alkaline composition, and an optional solvent), respectively. Reference to the reformulated birch bark pellets as including birch bark and various other components (e.g., an inert fibrous material, a mineral composition, an alkaline composition, and a solvent) is, however, acceptable and appropriate to those of skill in the art. As such, as used herein, “reformulated birch bark pellet” includes birch bark and various other components (e.g., an inert fibrous material, a mineral composition, an alkaline composition, and an optional solvent).

As used herein, the term “reflux” refers to the process of boiling a liquid in a vessel attached to a condenser so that the vapors continuously condense for reboiling.

As used herein, the term “ring debarker” refers to a machine in which the bark is peeled off the logs as they pass through the ring debarker.

As used herein, the term “Rosserhead debarker” refers to machine in which the logs are turned and a rotating debarking cutter head is pressed against the log.

As used herein, the term “rotary screen” refers to a screen that employs a rotational movement so that the particles on the screen move in a circular fashion.

As used herein, the term “screen” refers to a material woven from fine wires or made from any material with holes intended to block large particles from passing while allowing gases, liquids, and finer particles to pass. Typically, the material has a uniform pattern of holes.

As used herein, the term “shredder” refers to a machine that tears up objects into smaller pieces.

As used herein, the term “separating” refers to the process of removing solids from a mixture. The process can employ any technique known to those of skill in the art, e.g., decanting the mixture, filtering the solids from the mixture, or a combination thereof. Also, as used herein, the term “separating” refers to the process of classifying material into different particle sizes using, for example, a screen.

As used herein, the term “solvent” refers to a liquid that dissolves a solid, liquid, or gaseous solute, resulting in the formation of a solution. It is appreciated that those of skill in the art understand that the solvent should not chemically react with any of the starting materials or reagents present in the reaction mixture, to any significant degree, under the reaction conditions employed. For example, the solvent should not react with the alkaline present at the elevated temperatures typically employed during the heating step.

As used herein, the term “spent sunflower meal” refers to sunflower meal with the oil removed.

As used herein, the term “straight line screener” refers to a reciprocating screening machine that is used to separate and size materials. An example of this machine is the TEXAS SHAKER (a product of Screening Strategies, Dallas, Tex. 75223 USA).

As used herein, the term “trommel screen” refers to a type of screened cylinder that is used to separate materials by size.

As used herein, the term “water-immiscible solvent” refers to a solvent that is not miscible (i.e., not capable of mixing in all proportions) with water. Suitable specific water-immiscible solvents include, for example, alcohols such as butanol, pentanol, hexanol, and the like, aromatic hydrocarbons such as xylenes, o-xylene, m-xylene, p-xylene, toluene, benzene, and combinations thereof, chlorinated solvents such as chloroform and methylene chloride; as well as other organic solvents such as ethyl-tert-butyl ether and ethyl acetate.

As used herein, the term “washing” refers to the process of purifying a solid mass (e.g., crystals) by passing a liquid over and/or through the solid mass, as to remove soluble matter. The process includes passing a solvent, such as distilled water, over and/or through a precipitate obtained from filtering, decanting, or a combination thereof. For example, in one embodiment of the invention, washing includes contacting solids with water, vigorously stirring (e.g., for two hours), and filtering. The solvent can be water, can be an aqueous solvent system, or can be an organic solvent system. Additionally, the washing can be carried out with the solvent having any suitable temperature. For example, the washing can be carried out with the solvent having a temperature between about 0° C. and about 100° C.

In cases where compounds are sufficiently basic or acidic to form stable nontoxic acid or base salts, administration of the compounds as salts may be appropriate. Examples of pharmaceutically acceptable salts are organic acid addition salts formed with acids, which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts.

### DETAILED DESCRIPTION OF THE INVENTION

### Manufacture of Birch Bark Pellets

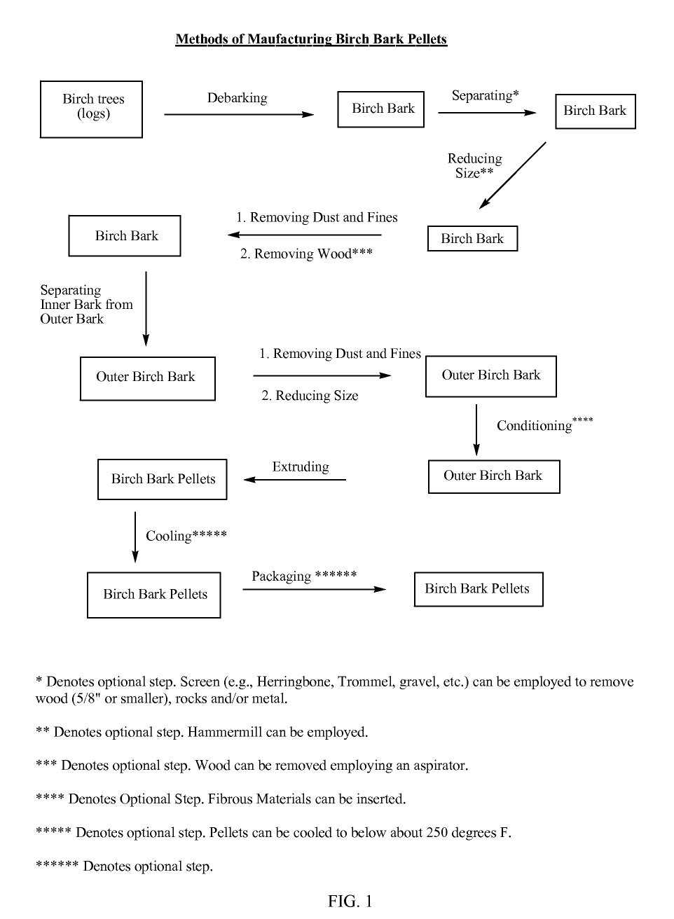

In one embodiment, the present invention affords a method for the manufacture of birch bark pellets. For example, birch trees may be cut into logs of appropriate size and the bark may be removed (see FIG. 1). This debarking step may be carried out employing a ring debarker, a drum debarker, or a Rosserhead debarker. The birch bark may undergo fragmentation by employing a chipper or a shredder to provide a combination of outer birch bark and inner birch bark. Next, the birch bark may optionally be separated from any debris (e.g., gravel) using a screen having openings of at least about ⅝ inch, preferably, having openings of about 1 inch or less. The debris may include rocks, metal, and wood having a diameter of ⅝ inch or smaller.

The bark may optionally be reduced in size using, for example, a hammermill. A hammermill is a machine that includes a steel drum containing a vertical or horizontal cross-shaped rotor on which pivoting hammers are mounted. The hammers are free to swing on the ends of the cross. The rotor is spun at a high speed inside the drum while material is fed into a feed hopper. The bark is impacted by the hammers on the ends of the rotating cross and thereby is shredded and expelled through screens in the drum.

Birch bark shreds less than about 10 mm in diameter can be used. More specifically, outer birch bark shreds less than about 6 mm in diameter, less than about 4 mm in diameter, or less than about 2 mm in diameter, can be used. Alternatively, birch bark pellets of about 5.0 mm in length by about 4 mm in diameter, or about 2.5 mm in length by about 2 mm in diameter can be used. In addition, bark pellets of about 0.25 kg/L to about 1.0 kg/L, or about 0.5 kg/L to about 0.7 kg/L can be used.

Next, the shredded birch bark may be separated from the fines (i.e., very small particles) and dust. The shredded birch bark may optionally be separated from any wood by employing an aspirator. The outer birch bark shred can be separated from the inner birch bark chunks using any suitable means. The separation can be accomplished by screening the mixture through a mesh having openings intermediate in size between the smaller inner bark chunks and the larger outer bark shreds. The smaller inner bark chunks fall through the screen and are separated from the outer bark. Typically, this separation may be carried out employing a Texas Shaker, a deck screen, or a trommel screen.

The mesh or screen can be a unit comprising one or more open spaces in a cord (i.e., string, rope, and the like), thread, or wire network in which the cords, threads or wires surround the spaces. Any mesh suitable to separate inner birch bark from outer birch bark can be employed. Typically, the mesh is a wire mesh containing openings of about ½ inch by ½ inch or smaller. For example, mesh can contain openings of about ¼ inch by about ¼ inch. Specifically, the size of the mesh can be about 20 mm by about 20 mm, about 10 mm by about 10 mm, or about 6 mm by about 6 mm. More specifically, the size of the mesh can be about 3 mm by about 3 mm.

Alternatively, the inner birch bark chunks and outer birch bark shreds may be separated with the use of an air classifier. As used herein, an “air classifier” is a device, which operates on the principle of the differing properties of the two components (e.g., inner and outer birch bark) in an air stream to effect a physical separation. Typically, the less dense outer bark travels a greater distance in the air stream than the more dense inner bark. The inner bark, along with other materials, falls rapidly from the stream of air. As a result, the inner birch bark and the outer birch bark can be separated. Once separated, the fines and dust are removed and the size of the outer birch bark may be reduced using a rotary cutter, a knife cutter, a hammermill, or any other suitable method of mechanical reduction. After separating outer birch bark from inner birch bark, outer birch bark of about 10 wt. % to about 45 wt. % based on initial birch bark content may typically be obtained and inner birch bark of about 55 wt. % to about 85 wt. % may typically be obtained.

The outer birch bark may optionally be conditioned, for example, by contacting the outer birch bark with steam, water, or a combination thereof.

The outer bark may also be conditioned with an inert fibrous material, a mineral composition, an alkaline composition, and a solvent.

Suitable inert fibrous materials may include, for example, inert fibrous plant material, inert fibrous animal material, inert fibrous mineral materials, inert fibrous polymeric material, and combinations thereof. Preferably, the inert fibrous plant material is material that has been de-oiled to remove the oils that are naturally present in the material.

Suitable inert de-oiled fibrous plant materials may include, for example, cellulose, corn husks, corn, corn stalks, cotton, coconut fibers, flax, grain, hemp, jute, lignin, oat hulls, oat straw, palm wastes, palm leaves, ramie, rice hulls, rice straw, sisal, spent sunflower meal (i.e., the material left after the oil is removed), soybean hulls, wheat straw, and combinations thereof. Preferably, the inert de-oiled fibrous material is ground up spent sunflower meal.

Suitable inert fibrous animal materials may include, for example, spider silk, sinew, catgut, hair, and combinations thereof. Suitable inert fibrous mineral materials may include, for example, fiberglass, metallic, carbon, and combinations thereof.

Suitable inert fibrous polymeric materials may include, for example, polyamide nylon, polyethylene terephthalate or polybutylene terephthalate polyester, phenol-formaldehyde, polyvinyl alcohol, polyvinyl chloride, polyolefins, acrylic, aromatic polyamides, polyethylene, elastomers, polyurethane, and combinations thereof.

Suitable mineral compositions may include, for example, metal carbonates, and combinations thereof. Preferably, the metal carbonate is lime (i.e., ground up limestone).

Suitable alkaline compositions may include, for example, an alkali metal hydroxide, an alkaline earth metal hydroxide, or a combination thereof. Suitable alkali metal hydroxides may include, for example, sodium hydroxide, potassium hydroxide, lithium hydroxide, rubidium hydroxide, caesium hydroxide, francium hydroxide, and combinations thereof. Preferably, the alkali metal hydroxide is sodium hydroxide. Suitable alkaline earth metal hydroxides may include, for example, magnesium hydroxide, calcium hydroxide, and combinations thereof.

Suitable solvents include, for example, an aqueous solvent, an organic solvent, or a combination thereof. Preferably, the solvent is water.

The outer bark may preferably be conditioned with spent sunflower meal, lime, sodium hydroxide, and water.

The outer birch bark may also be conditioned with other optional adjuvants in addition to the ingredients described above. Suitable adjuvants may include, for example, fillers, minerals, and other ingredients that will be apparent to those skilled in the art.

After the optional conditioning step, the outer birch bark may be extruded employing a die having an orifice with a diameter of about 1/32 inch to about 2 inches, preferably about ⅛ inch to about ¾ inch. Typically, the average temperature of the extruder die may be from about 75° F. to about 300° F., preferably from about 100° F. to about 250° F. After extrusion, the birch bark pellets may be cooled to about 105° F. to about 150° F., preferably about 125° F. to about 130° F. The cooled bark pellets may be packaged for shipment.

Typically, at least about 15 kg of the birch bark pellets are obtained per about 100 kg of birch bark employed.

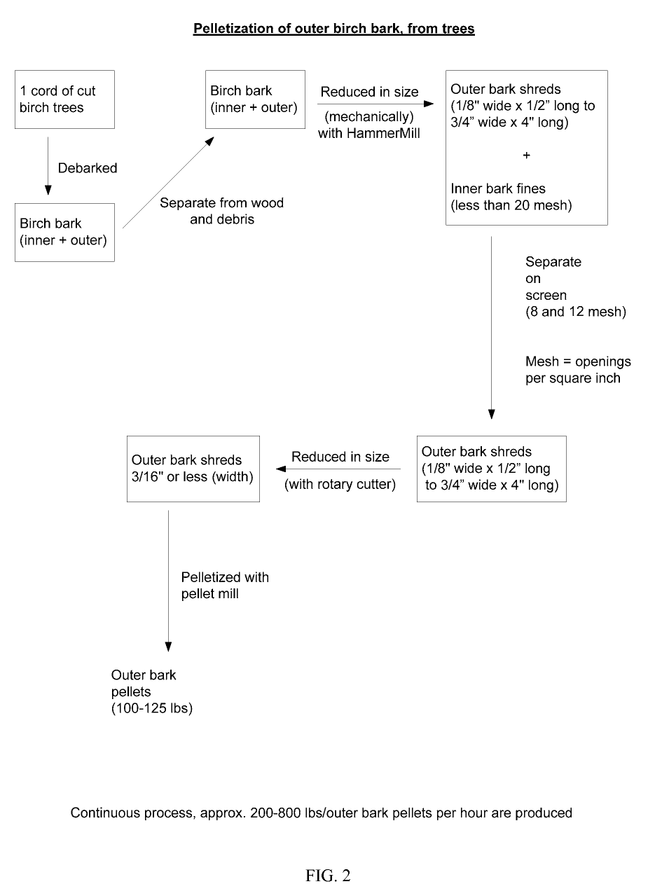

In another embodiment, the present invention affords a method for the manufacture of birch bark pellets (see FIG. 2). For example, one cord (i.e., about 128 cubic feet) of cut trees may be debarked to afford a combination of inner and outer birch bark. The combination of inner and outer birch bark may be separated from wood and debris by the methods described above. The combination of inner and outer birch bark may be mechanically reduced in size by employing, for example, a hammermill. Typically, this affords a combination of outer bark shreds (⅛ inches wide by ½ inches long to ¾ inches wide by 4 inches long) and inner bark fines (less than 20 mesh). The combination of inner and outer birch bark may be separated on a screen (e.g., 8 and 12 mesh) to afford outer bark shreds. The outer bark shreds are typically reduced in size with a rotary cutter to afford outer bark shreds of 3/16 inches or less (width). The outer bark shreds are then pelletized with a pellet mill to afford approximately 100-125 pounds (lbs.) of outer bark pellets. Typically in this continuous process, approximately 200-800 lbs. of outer pellets are produced per hour.

### Composition of the Birch Bark Pellets

In another embodiment, the present invention affords a birch bark pellet.

Typically, a birch bark pellet may include at least about 30 wt. % outer birch bark, preferably at least about 40 wt. % outer birch bark, preferably at least about 50 wt. % outer birch bark, preferably at least about 60 wt. % outer birch bark, and preferably at least about 70 wt. % outer birch bark. Typically, a birch bark pellet may include less than about 30 wt. % inner birch bark, preferably less than about 20 wt. % inner birch bark, and preferably less than about 10 wt. % inner birch bark. Typically, a birch bark pellet may also include less than about 30 wt. % wood, preferably less than about 20 wt. % wood, and preferably less than about 10 wt. % wood.

Typically, a birch bark pellet may also include up to about 10 wt. % free (extractable) betulin, preferably up to about 15 wt. % free (extractable) betulin, and preferably up to about 20 wt. % free (extractable) betulin. Typically, a birch bark pellet may also include greater than about 5 wt. % free (extractable) betulin. Typically, a birch bark pellet may have a density of at least 0.2 gm/cm³, preferably a density of at least 0.4 gm/cm³. Typically, the volume of the birch bark pellet may be at least about 0.40 cm³ per pellet. Typically, a birch bark pellet may include less than about 45 wt. % water, preferably less than about 30 wt. % water, preferably less than about 15 wt. % water, and preferably less than about 5 wt. % water. Typically, the diameter of the birch bark pellets may be from about 1/32 inch to about 2 inches, preferably about ⅛ inch to about ¾ inch.

If desired, a birch bark pellet may include at least about 5 wt. % inert fibrous material, preferably at least about 15 wt. % inert fibrous material, preferably at least about 20 wt. % inert fibrous material, preferably at least about 25 wt. % inert fibrous material, preferably at least about 30 wt. % inert fibrous material, preferably at least about 35 wt. % inert fibrous material, and preferably at least about 40 wt. % inert fibrous material.

If desired, a birch bark pellet may include at least about 0.5 wt. % mineral composition, preferably at least about 1.5 wt. % mineral composition, preferably at least about 2.0 wt. % mineral composition, preferably at least about 2.5 wt. % mineral composition, preferably at least about 3.0 wt. % mineral composition, preferably at least about 3.5 wt. % mineral composition, and preferably at least about 4.0 wt. % mineral composition.

If desired, a birch bark pellet may include at least about 0.1 wt. % alkaline composition, preferably at least about 0.3 wt. % alkaline composition, preferably at least about 0.5 wt. % alkaline composition, preferably at least about 0.7 wt. % alkaline composition, preferably at least about 0.9 wt. % alkaline composition, preferably at least about 2.0 wt. % alkaline composition, and preferably at least about 5.0 wt. % alkaline composition.

If desired, a birch bark pellet may include at least about 5 wt. % optional solvent, preferably at least about 15 wt. % optional solvent, preferably at least about 25 wt. % optional solvent.

If desired, a birch bark pellet may include at least about 0.1 wt. % optional adjuvants, preferably at least about 1 wt. % optional adjuvants, preferably at least about 2 wt. % optional adjuvants, preferably at least about 5 wt. % optional adjuvants, preferably at least about 10 wt. % optional adjuvants, preferably at least about 20 wt. % optional adjuvants, and preferably at least about 30 wt. % optional adjuvants.

### Manufacture of Reformulated Birch Bark Pellets

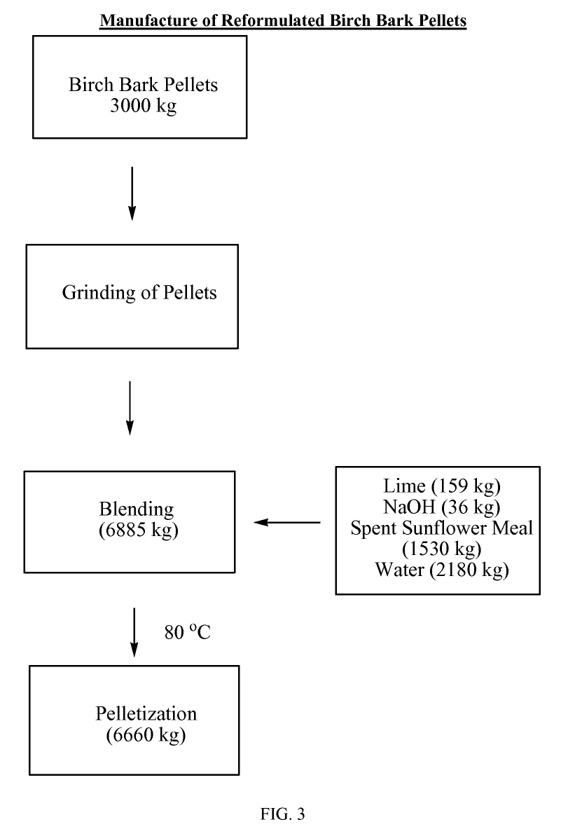

In another embodiment, the present invention affords a method for the reformulation of birch bark pellets (see FIG. 3). For example, birch bark pellets, produced by any of the above methods, are ground up and blended with an inert fibrous material, an mineral composition, an alkaline composition, and an optional solvent.

Suitable inert fibrous materials may include, for example, inert fibrous plant material, inert fibrous animal material, inert fibrous mineral materials, inert fibrous polymeric material, and combinations thereof. Preferably, the inert fibrous plant material is material that has been de-oiled to remove the oils that are naturally present in the material.

Suitable inert de-oiled fibrous plant materials may include, for example, cellulose, corn husks, corn, corn stalks, cotton, coconut fibers, flax, grain, hemp, jute, lignin, oat hulls, oat straw, palm wastes, palm leaves, ramie, rice hulls, rice straw, sisal, spent sunflower meal (i.e., the material left after the oil is removed), soybean hulls, wheat straw, and combinations thereof. Preferably, the inert de-oiled fibrous material is ground up spent sunflower meal.

Suitable inert fibrous animal materials may include, for example, spider silk, sinew, catgut, hair, and combinations thereof. Suitable inert fibrous mineral materials may include, for example, fiberglass, metallic, carbon, and combinations thereof.

Suitable inert fibrous polymeric materials may include, for example, polyamide nylon, polyethylene terephthalate or polybutylene terephthalate polyester, phenol-formaldehyde, polyvinyl alcohol, polyvinyl chloride, polyolefins, acrylic, aromatic polyamides, polyethylene, elastomers, polyurethane, and combinations thereof.

Suitable mineral compositions may include, for example, metal carbonates, and combinations thereof. Preferably, the metal carbonate is lime (i.e., ground-up limestone).

Suitable alkaline compositions may include, for example, an alkali metal hydroxide, an alkaline earth metal hydroxide, or a combination thereof. Suitable alkali metal hydroxides may include, for example, sodium hydroxide, potassium hydroxide, lithium hydroxide, rubidium hydroxide, caesium hydroxide, francium hydroxide, and combinations thereof. Preferably, the alkali metal hydroxide is sodium hydroxide. Suitable alkaline earth metal hydroxides may include, for example, magnesium hydroxide, calcium hydroxide, and combinations thereof.

Suitable solvents include, for example, an aqueous solvent, an organic solvent, or a combination thereof. Preferably, the solvent is water.

Preferably, the outer bark may be blended with spent sunflower meal, lime, sodium hydroxide, and water.

The outer birch bark may also be conditioned with other optional adjuvants in addition to the ingredients described above. Suitable adjuvants may include, for example, fillers, minerals, and other ingredients that will be apparent to those skilled in the art.

After blending, the mixture may be heated to at least about 80° C. and extruded through a die to afford reformulated birch bark pellets.

### Composition of the Reformulated Birch Bark Pellets

Typically, a reformulated birch bark pellet may include at least about 15 wt. % birch bark, preferably at least about 30 wt. % birch bark, preferably at least about 45 wt. % birch bark, preferably at least about 60 wt. % birch bark, preferably at least about 75 wt. % birch bark, and preferably at least about 90 wt. % birch bark.

Typically, a reformulated birch bark pellet may include at least about 30 wt. % outer birch bark, preferably at least about 40 wt. % outer birch bark, preferably at least about 50 wt. % outer birch bark, preferably at least about 60 wt. % outer birch bark, and preferably at least about 70 wt. % outer birch bark.

Typically, a reformulated birch bark pellet may include less than about 30 wt. % inner birch bark, preferably less than about 20 wt. % inner birch bark, and preferably less than about 10 wt. % inner birch bark. Typically, a reformulated birch bark pellet may also include less than about 30 wt. % wood, preferably less than about 20 wt. % wood, and preferably less than about 10 wt. % wood.

Typically, a reformulated birch bark pellet may also include up to about 20 wt. % free (extractable) betulin. Typically, a reformulated birch bark pellet may also include greater than about 10 wt. % free (extractable) betulin. Typically, a reformulated birch bark pellet may have a density of at least 0.2 gm/cm³, preferably a density of at least 0.4 gm/cm³. Typically, a reformulated birch bark pellet may include less than about 45 wt. % water, preferably less than about 30 wt. % water, preferably less than about 15 wt. % water, and preferably less than about 5 wt. % water.

Typically, the diameter of the reformulated birch bark pellets may be from about 1/32 inch to about 2 inches, preferably about ⅛ inch to about ¾ inch.

Typically, a reformulated birch bark pellet may include at least about 5 wt. % inert fibrous material, preferably at least about 15 wt. % inert fibrous material, preferably at least about 20 wt. % inert fibrous material, preferably at least about 25 wt. % inert fibrous material, preferably at least about 30 wt. % inert fibrous material, preferably at least about 35 wt. % inert fibrous material, and preferably at least about 40 wt. % inert fibrous material.

Typically, a reformulated birch bark pellet may include at least about 0.5 wt. % mineral composition, preferably at least about 1.5 wt. % mineral composition, preferably at least about 2.0 wt. % mineral composition, preferably at least about 2.5 wt. % mineral composition, preferably at least about 3.0 wt. % mineral composition, preferably at least about 3.5 wt. % mineral composition, and preferably at least about 4.0 wt. % mineral composition.

Typically, a reformulated birch bark pellet may include at least about 0.1 wt. % alkaline composition, preferably at least about 0.3 wt. % alkaline composition, preferably at least about 0.5 wt. % alkaline composition, preferably at least about 0.7 wt. % alkaline composition, preferably at least about 0.9 wt. % alkaline composition, preferably at least about 2.0 wt. % alkaline composition, and preferably at least about 5.0 wt. % alkaline composition.

Typically, a reformulated birch bark pellet may include at least about 5 wt. % optional solvent, preferably at least about 15 wt. % optional solvent, preferably at least about 25 wt. % optional solvent, preferably at least about 30 wt. % optional solvent, preferably at least about 35 wt. % optional solvent, preferably at least about 45 wt. % optional solvent, and preferably at least about 50 wt. % optional solvent.

Typically, a reformulated birch bark pellet may include at least about 0.1 wt. % optional adjuvants, preferably at least about 1 wt. % optional adjuvants, preferably at least about 2 wt. % optional adjuvants, preferably at least about 5 wt. % optional adjuvants, preferably at least about 10 wt. % optional adjuvants, preferably at least about 20 wt. % optional adjuvants, and preferably at least about 30 wt. % optional adjuvants.

### Extraction of Birch Bark Pellets

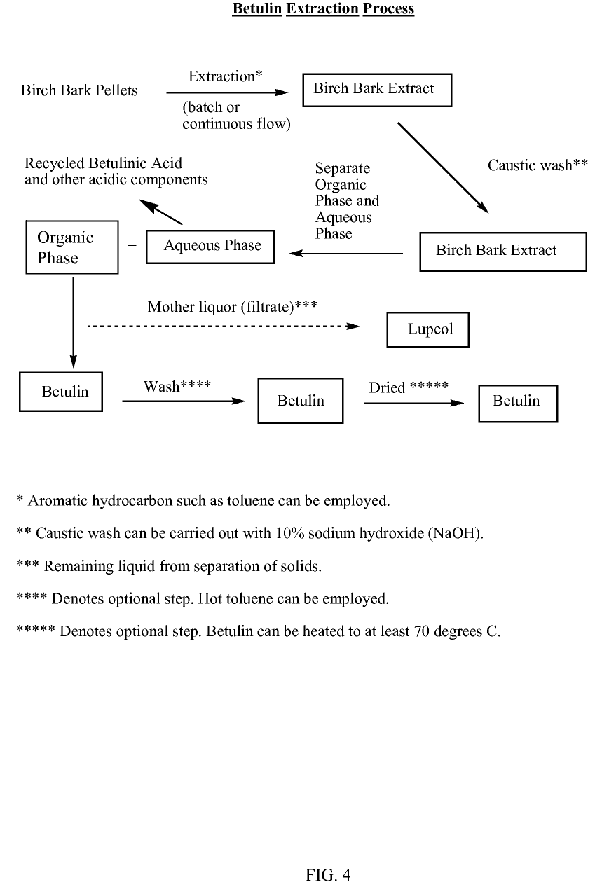

In one embodiment, the present invention affords a method for the extraction of betulin (see FIG. 4). For example, birch bark pellets may be extracted with an aromatic hydrocarbon (e.g., toluene) in either a batch or continuous process to afford the birch bark extract. The birch bark extract may be washed with aqueous sodium hydroxide. The organic phase (e.g., birch bark extract in toluene) may be then separated from the aqueous phase (e.g., aqueous sodium hydroxide). The aqueous phase contains betulinic acid and other acidic components. The organic phase may be allowed to settle to allow the betulin to crystallize out. The organic phase may be filtered to remove the betulin. The filtrate may be subsequently processed to afford lupeol. The betulin crystals are optionally washed with hot toluene and optionally dried to at least 70° C.

### Extraction of the Reformulated Birch Bark Pellets

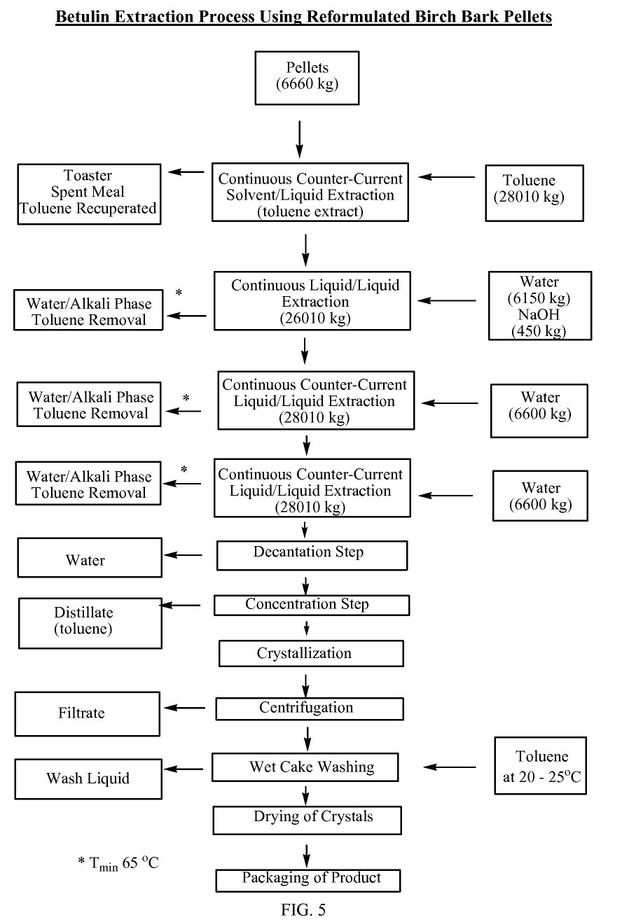

In one embodiment, the present invention affords a method for the extraction of betulin (see FIG. 5). For example, reformulated birch bark pellets that include outer bark, spent sunflower meal, lime, sodium hydroxide, and optional water may be extracted with an aromatic hydrocarbon (e.g., toluene) in a continuous countercurrent solid/liquid extraction to afford the birch bark extract. The waste from this step (e.g., spent sunflower meal and toluene) is recycled.

The birch bark extract may be subjected to at least one or more continuous liquid/liquid extraction with aqueous sodium hydroxide followed by at least one or more continuous counter-current liquid/liquid extractions with water. Preferably, the birch bark extract may be subjected to one continuous liquid/liquid extraction at a temperature of at least 65° C. with aqueous sodium hydroxide followed by two continuous counter-current liquid/liquid extractions at a temperature of at least 65° C. with water.

The excess water may be decanted from the birch bark extract. The birch bark extract may be concentrated by evaporation and the betulin may be allowed to crystallize. The birch bark extract with the crystallized betulin may be subjected to centrifugation, the filtrate may be removed and the wet cake of betulin may be washed with toluene. The toluene may be removed and the betulin may be dried. After drying, the betulin may be packaged.

### Manufacture of Betulin, Betulinic Aldehyde-3-Acetate, and Betulinic Acid

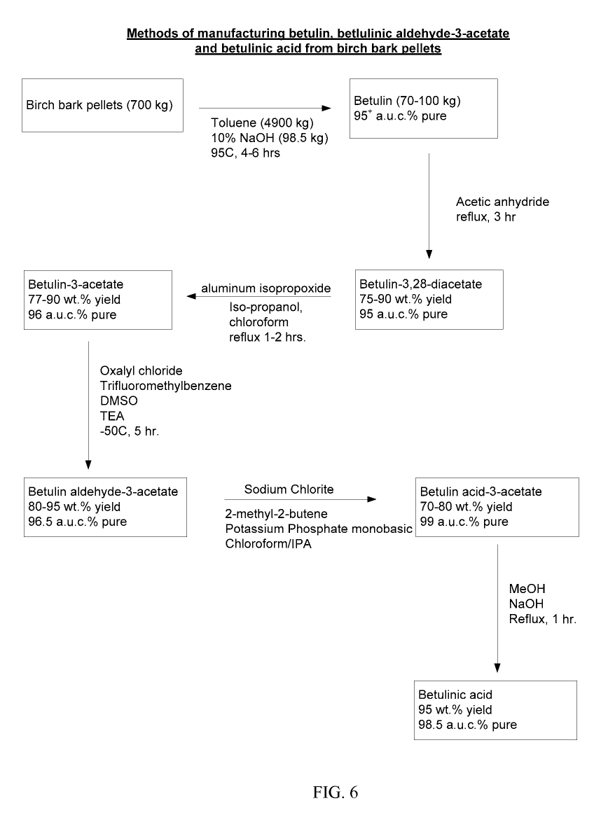

In one embodiment, the present invention affords a method for the manufacture of betulin, betulinic aldehyde-3-acetate, and betulinic acid from birch bark pellets (see FIG. 6). For example, birch bark pellets are extracted with toluene and 10 wt. % aqueous sodium hydroxide at elevated temperatures for several hours to afford betulin. The betulin may be refluxed with acetic anhydride for several hours to afford betulin-3,28-diacetate. The betulin-3,28-diacetate may be reacted with aluminum isopropoxide at elevated temperatures for several hours to afford betulin-3-acetate. The betulin-3-acetate may be reacted with oxalyl chloride, trifluoromethylbenzene, dimethyl sulfoxide (DMSO), and triethylamine (TEA) at reduced temperatures for several hours to afford betulinic aldehyde-3-acetate. The betulinic aldehyde-3-acetate may be reacted with sodium chlorite, 2-methyl-2-butene, and potassium phosphate monobasic to afford betulinic acid-3-acetate. The betulinic acid-3-acetate may be reacted with sodium hydroxide at elevated temperatures for several hours to afford betulinic acid.

The following Examples are illustrative of the above invention. One skilled in the art will readily recognize that the techniques and reagents described in the Examples suggest many other ways in which the present invention could be practiced. It should be understood that many variations and modifications may be made while remaining within the scope of the invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

### Example 1

### Birch Bark Processing

A bark batch feeder (6,000 pound per hour or 1,200 cubic feet per hour) was loaded with a bucket loader from a bark pile. The feeder provided an even flow of bark to a belt conveyor going to the hammermill. The bark lay down area included a fines blower and fines van parked on the site and a bag house, which was required to control dust.

A hammermill (6,000 pound per hour or 1,200 cubic feet per hour) was fed by a belt conveyor. It was discharged by a fan operated by same drive motor as the hammermill. The fan moved shredded bark and fines to a cyclone with rotary airlock. The rotary airlock discharged to a screw auger conveyor to take inner and outer bark to 2 deck screens (mesh), which will handle 6,000 pounds per hour. A fines auger took inner bark “rejects” (4,800 pounds per hour) from the screen to a blower. A blower discharged inner bark fines into a van trailer.

Screened outer bark “accepts” (1,200 pounds per hour) were discharged onto a screw auger conveyor to feed the rotary cutter. The rotary cutter was fed from this conveyor (1,200 pounds per hour) and granulated outer bark was moved by air to a cyclone with a rotary airlock, which discharged product into a surge bin. The surge bin with its 55 cubic feet capacity (approx 30 minutes run time when full) was used to feed the pellet mill feeder/conditioner by auger conveyor. The surge bin had a full and low level control switch. A screw auger conveyor was used to move the granulated outer bark to the feeder-conditioner.

A pellet mill was fed through the feeder/conditioner. Pellets from the pellet mill were cooled and fines removed by a vibrating screen conveyor. This conveyor was connected to an 18-inch belt conveyor, which carried pellets to super sacks.

Birch bark was run through the hammermill with a shredder (slotted) screen. Outer bark was separated from the inner bark with a deck screen (mesh). A rotary cutter was used to granulate the outer bark to a size that could be run through a pellet mill.

### Example 2

### Density of Birch Bark Pellets

The density of representative birch bark pellets described herein can be found in Table 1 below.

TABLE 1

| Length | Diameter | Volume | Weight | Density | | Std | |
|---|---|---|---|---|---|---|---|
| (cm) | (cm) | (cc) | (g) | (g/cc) | Mean | dev | RSD |
| Pellets after wood removal system installed | | | | | | | |
| 1.50 | 0.635 | 0.475 | 0.4988 | 1.051 | | | |
| 1.70 | 0.635 | 0.538 | 0.6032 | 1.121 | | | |
| 2.00 | 0.635 | 0.633 | 0.6808 | 1.075 | | | |
| 1.30 | 0.635 | 0.411 | 0.4350 | 1.057 | | | |
| 1.45 | 0.635 | 0.459 | 0.5113 | 1.114 | | | |
| 1.75 | 0.635 | 0.554 | 0.5686 | 1.026 | | | |
| 2.05 | 0.635 | 0.649 | 0.6776 | 1.044 | | | |
| 1.60 | 0.635 | 0.506 | 0.5312 | 1.049 | | | |
| 2.00 | 0.635 | 0.633 | 0.6370 | 1.006 | | | |
| 2.20 | 0.635 | 0.696 | 0.7408 | 1.064 | 1.061 | 0.036 | 3.35% |
| Pellets before wood removal system installed | | | | | | | |
| 1.70 | 0.635 | 0.538 | 0.6650 | 1.236 | | | |
| 1.95 | 0.635 | 0.617 | 0.7598 | 1.231 | | | |
| 1.55 | 0.635 | 0.491 | 0.5790 | 1.180 | | | |
| 1.80 | 0.635 | 0.570 | 0.6842 | 1.201 | | | |
| 2.10 | 0.635 | 0.665 | 0.8061 | 1.213 | | | |
| 1.50 | 0.635 | 0.475 | 0.5735 | 1.208 | | | |
| 1.90 | 0.635 | 0.601 | 0.7420 | 1.234 | | | |
| 1.30 | 0.635 | 0.411 | 0.4886 | 1.187 | | | |
| 1.95 | 0.635 | 0.617 | 0.7434 | 1.204 | | | |
| 0.90 | 0.635 | 0.285 | 0.3565 | 1.251 | 1.215 | 0.023 | 1.88% |

### Example 3

### Isolation of Betulin from Birch Bark Pellets

TABLE 2

| Materials: | Quantity: |
|---|---|
| Birch Bark Pellets | |
| containing typically | |
| 15-25% w/w Betulin | |
| Toluene | 7 kg/kg Pellets |
| 10% Aqueous NaOH Solution | 1 kg/5 kg of Toluene |
| Equipment | Type |
| | Littleford Day Extractor |
| | Reactor/Settler with Recirculation Loop |
| | Neutsche Type Filter |

This process was performed in all stainless steel equipment. All process lines carrying extract were jacketed and maintained at a minimum temperature of 95° C.

The process described employed a 1,000 gallon Littleford Day Extractor, equipped with a horizontal plough type agitator, suitable solids feed hopper system, and a dry solids discharge system.

Approximately 700 kg of birch bark pellets were charged to the extractor, via the integral hopper system, and the system was blanketed with nitrogen. The system was heated to between 85 and 105° C., preferably 95° C. 4,900 kg of toluene preheated to 95° C. was fed to the extractor, under nitrogen pressure and the system was left pressurized above atmospheric pressure. The system was left to extract for approximately 3-5 hours, preferably 4 hours. The plough agitator was rotated for 1 minute every 30 minutes to ensure even heat distribution. The extracted materials in toluene were removed from the extractor, via the integral filter under nitrogen top pressure, and the resultant clear extract was delivered to the caustic wash tank.

An additional 1400 kg of fresh preheated toluene was added to the extractor and left to contact the pellets for an additional time of at least 20 minutes, with the plough agitator being rotated every 5 minutes. This additional toluene was removed from the extractor, by nitrogen top pressure, via the integral filter, and sent to the toluene surge drum. The extractor was placed in solvent removal mode, where a recirculating flow of hot nitrogen was used to dry the pellets of all residual solvent prior to discharge of the spent pellets. The extract in the caustic wash tank was recirculated via an in-line mixer where it was contacted with a recirculating flow of hot 10% caustic solution.

On re-entering the vessel the two phases were separated and recirculated via appropriate pumps. This neutralization step continued to allow for a minimum 3 recirculations of the entire extract batch. The aqueous phase was withdrawn for disposal. The clarified extract was sent to a Neutsche type filter where it was allowed to cool below 80° C. The extracted betulin was allowed to crystallize in the vessel.

The extract was filtered in the Neutsche filter and the mother liquor sent to lupeol recovery. The crystallized betulin, typically 70-100 kg, was washed with a minimum of 200 kg of toluene, heated to a minimum of 70° C. The toluene wash was sent to the toluene surge tank for use in the next extraction batch. The crystallized, washed betulin was dried in the Neutsche Filter. The betulin was discharged to drying trays and sent to the drier for residual solvent removal.

This process can be performed in a continuous belt extractor, at a temperature of about 95° C. Utilizing (outer) birch bark in the form of pellets, and utilizing toluene as the solvent for extraction, the yield was increased from about 60 wt. % to about 85 wt. %. Additionally, the amount of solvent employed was reduced from about 7 kg/kg of (outer) birch bark to about 5 kg/kg of (outer) birch bark.

### Example 4

### Isolation of Betulin from Reformulated Birch Bark Pellets

The following process was carried out using a continuous belt extraction process. Approximately 18.850 kg of birch bark pellets were reformulated, using about 1% NaOH, 5% lime, 50% spent sunflower meal (from food oil extraction), and 60% water. The reformulated pellets were 12 mm and were stable to allow good percolation rates of solvent on the moving bed.

Pellets were continuously fed to the belt extractor and extracted with toluene at 75° C. Solvent to feed ratio (on the basis of the original birch bark pellets) was about 10:1. The extraction time on the belt was about 5 hours and the toluene solvent was pumped countercurrent to the belt flow. Extraction efficiency was about 95%, based on residual betulin in the spent pellets.

The extract, containing about 1% betulin was passed through 3 stages of liquid/liquid extraction where it was first extracted against a 7.5% NaOH solution, at a solvent:caustic ratio of 5:1. Lower caustic concentrations were found not to remove all of the extracted acidic components and higher concentrations degraded the recovered betulin purity.

This caustic wash was followed by 2 stages of liquid/liquid extraction against water using a solvent:water ratio of about 4:1. These multiple treatments lowered the residue on ignition, a good measure of the residual inorganic content, to below 0.1%, against the previous levels of about 1-2%.

Following the liquid/liquid steps the extract was decanted to remove trace water because a residual water content above 1% results in a much lower purity of product betulin (92% versus 97%). Toluene was evaporated to concentrate the extract to about 5% betulin, prior to crystallization. The concentrated solvent was fed to a continuous cooler where the temperature was lowered to about 10° C. Higher concentrations of the extract were found to yield lower betulin purity, and lower temperatures had a similar effect. Crystallization time appeared to have no effect on purity, but did marginally affect yield.

The extract was fed to a centrifuge for betulin recovery, washed with toluene at about 20° C. and dried at about 85° C. and 50 millibar (mbar) for at least 6 hours to reduce residual toluene to below ICH (International Conference on Harmonisation of Technical Requirements for Registration of Pharmaceuticals for Human Use) guidelines of 870 parts per million (ppm).

This process resulted in about 1675 kg of betulin with a purity of about 98% w/w by HPLC (High-performance liquid chromatography).

Betulin purity from this process (97%) is higher than for the batch process (95%).

The residue on ignition test result (an indication of the inorganic content) for the betulin from this process was less than 0.1% versus 1-2% for the batch produced product. This is a major factor for the subsequent use of the betulin as a raw material for downstream synthesis of derivatives.

Processing in the continuous process is far less complex and requires far less overall processing time than the batch process. The continuous process requires less than 20 hours including drying, versus 30 plus hours for the batch process.

Recovery and recycle of spent toluene is also far higher with the continuous process, as there are significantly smaller amounts of resins and other materials extracted at the lower temperatures of extraction, and hence greater recovery of spent toluene is possible (90%+versus 70% for the batch process).

The continuous process also involves fewer issues associated with the handling of toluene vapors, due to the much lower vapor pressure at 75° C. than at the 90-95° C. used in the batch process.

The betulin produced by this continuous process mitigates the problems associated with processing the batch produced betulin to betulinic acid and other derivates. This is believed to be associated with the much lower residue on ignition levels, and presumably with the higher HPLC purity.

What is claimed is:

1. A method of manufacturing birch bark pellets, the method comprising:
(a) debarking a birch tree to provide birch bark;
(b) separating the inner birch bark from the outer birch bark;
(c) reducing the size of the outer birch bark;
(d) conditioning the outer birch bark; and
(e) extruding the outer birch bark, effective to provide birch bark pellets.

2. The method of claim 1, further comprising, after the debarking the birch tree to provide birch bark in step (a), reducing the size of the birch bark.

3. The method of claim 1, wherein the separating the inner birch bark from the outer birch bark in step (b) comprises subjecting the birch bark to fragmentation to provide a combination of outer birch bark and inner birch bark, and separating the outer birch bark from the inner birch bark by passing the inner birch bark through a mesh effective to separate the outer birch bark from the inner birch bark.

4. The method of claim 1, wherein the conditioning the outer birch bark in step (d) comprises contacting the outer birch bark with at least one of steam and water.

5. The method of claim 1, wherein the extruding the outer birch bark in step (e) is carried out employing a die having an orifice with a diameter of about ⅛ inch to about ¾ inch.

6. The method of claim 1, wherein the extruding the outer birch bark in step (e) is carried out employing a die having an average temperature of about 100° F. to about 250° F.

7. The method of claim 1, further comprising:
(f) grinding the birch bark pellets;
(g) blending the ground birch bark pellets with an inert fibrous material, a mineral composition, an alkaline composition, and an optional solvent to afford a blended birch bark composition; and
(h) extruding the blended birch bark composition to provide reformulated birch bark pellets.

8. A reformulated birch bark pellet comprising ground birch bark with an inert fibrous material, a mineral composition, an alkaline composition, and an optional solvent.

9. The reformulated birch bark pellet of claim 8, wherein the inert fibrous material comprises de-oiled fibrous plant material.

10. The reformulated birch bark pellet of claim 8, wherein the mineral composition comprises a metal carbonate.

11. The reformulated birch bark pellet of claim 8, wherein the alkaline composition comprises an alkali metal hydroxide, an alkaline earth metal hydroxide, or a combination thereof.

12. The reformulated birch bark pellet of claim 8, wherein the optional solvent comprises an aqueous solvent, an organic solvent, or a combination thereof.

13. The reformulated birch bark pellet of claim 8, wherein the reformulated birch bark pellet comprises at least about 30 wt. % outer birch bark.

14. The reformulated birch bark pellet of claim 8 that comprises less than about 6 wt. % inner birch bark.

15. The reformulated birch bark pellet of claim 8 that comprises greater than about 2 wt. % free (extractable) betulin.

16. A method for obtaining a natural product from birch bark pellets, the method comprising:
(a) contacting birch bark pellets with an organic solvent to provide a birch bark extract;
(b) contacting the birch bark extract with an aqueous base to provide an organic phase and an aqueous phase;
(c) separating the organic phase from the aqueous phase; and
(d) separating solids from the organic phase to provide the natural product.

17. The method of claim 16, wherein the natural product comprises betulin, lupeol, or a combination thereof.

18. The method of claim 16, wherein the contacting of the birch bark pellets with the organic solvent is carried out at a temperature of about 65° C. to about 105° C., wherein the organic solvent comprises toluene.

19. The method of claim 16, further comprising, after the contacting the birch bark extract with the aqueous base in step (b), separating the birch bark extract and the aqueous base, and crystallizing betulin from the extract, wherein contacting the birch bark extract with the aqueous base is carried out at a temperature and for a period of time effective to hydrolyze natural esters of the natural product.

20. The method of claim 16, further comprising, after the contacting the birch bark extract with the aqueous base in step (b), separating the birch bark extract and the aqueous base, washing the birch bark extract at least one or more times with water, decanting the birch bark extract to remove water, reducing the organic solvent from the birch bark extract by evaporation, cooling the birch bark extract, crystallizing betulin from the birch bark extract, and washing the betulin with toluene, wherein the birch bark pellets are reformulated birch bark pellets comprising ground birch bark with an inert fibrous material, a mineral composition, an alkaline composition, and an optional solvent.
